(19) United States
(12) Patent Application Publication
(10) Pub. No.: US 2018/0216148 A1
(43) Pub. Date: Aug. 2, 2018

(54) COMPOSITE CELLULOSE HYDROGELS AND METHODS OF MAKING AND USE THEREOF

(71) Applicants: Chelsea Elisabeth Casper, Austin, TX (US); BOARD OF REGENTS, THE UNIVERSITY OF TEXAS SYSTEM, Austin, TX (US)

(72) Inventors: R. Malcolm Brown, JR., Manor, TX (US); Sarah J. Pfeffer, Austin, TX (US); Chelsea Elisabeth Casper, Austin, TX (US)

(21) Appl. No.: 15/884,718

(22) Filed: Jan. 31, 2018

Related U.S. Application Data
(60) Provisional application No. 62/452,411, filed on Jan. 31, 2017.

(51) Int. Cl. C12P 19/04; A61L 27/52; A61L 27/20; A61K 8/04; A61L 15/28; A61K 8/73; A61L 15/60

(52) U.S. Cl. CPC C12P 19/04; A61L 27/52; A61L 27/20; A61L 15/60; A61L 15/28; A61K 8/731; A61K 8/042

(57) ABSTRACT

Disclosed herein are methods of making composite cellulose hydrogels, the methods comprising providing a cellulose synthesizing microbe; and culturing the cellulose synthesizing microbe in a composition comprising greater than 1% of a cellulose derivative, thereby forming the composite cellulose hydrogel.

### CROSS-REFERENCE TO RELATED APPLICATIONS

This application claims the benefit of priority to U.S. Provisional Application No. 62/452,411, filed Jan. 31, 2017, which is hereby incorporated herein by reference in its entirety.

### BACKGROUND

Cellulose is the most abundant biopolymer on earth and is produced by a variety of organisms, including plants, algae, tunicates, colorless protists, as well as photosynthetic and heterotrophic bacteria (Brown R M Jr. J Cell Sci Suppl. 1985, 2, 13-32; Ross P et al. Microbiol Rev. 1991, 55,35-58; Blanton R L et al. Proc Natl Acad Sci USA. 2000, 97, 2391-2396; Kimura T. Jpn. Soc. Composite materials, Applications of Composite Materials. 2001, 828-835). Certain bacterial strains can also produce cellulose, and each bacterial strain will create different characteristics for the cellulose material (Czaja W et al. Cellulose, 2004, 11, 403-411).

There is a need for a more affordable, longer-lasting injectable material for soft tissue reconstruction due to defects caused by disease, trauma, and aging. Additionally, topical skin repair products are in high demand. Cosmetics and skincare is projected to become a $265 billion a year industry due to GDP growth. Products available today have a vast variety of effectiveness and cost per product; many of the products available today are ineffective and costly. Market demand is high for effective, science driven products. The compositions and methods discussed herein address these and other needs.

### SUMMARY

In accordance with the purposes of the disclosed compositions and methods, as embodied and broadly described herein, the disclosed subject matter relates to compositions and methods of making and use thereof. More specifically, composite cellulose hydrogels and methods of making and use thereof are described herein.

Additional advantages of the disclosed compositions and methods will be set forth in part in the description which follows, and in part will be obvious from the description. The advantages of the disclosed compositions will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The accompanying figures, which are incorporated in and constitute a part of this specification, illustrate several aspects of the disclosure, and together with the description, serve to explain the principles of the disclosure.

FIG. 1 is a bright field image of a bacterial cellulose/carboxymethyl cellulose hydrogel composite synthesized using K. Hansenii NQ4 with the addition of 1% medium viscosity carboxymethyl cellulose (scale bar 100 μm).

FIG. 2 is a first order red polarized light image of a bacterial cellulose/carboxymethyl cellulose hydrogel composite synthesized using K. Hansenii NQ4 with the addition of 1% medium viscosity carboxymethyl cellulose (scale bar 100 μm).

FIG. 3 is a polar extinction image of a bacterial cellulose/carboxymethyl cellulose hydrogel composite synthesized using K. Hansenii NQ4 with the addition of 1% medium viscosity carboxymethyl cellulose (scale bar 100 μm).

FIG. 4 is a bright field image of a bacterial cellulose/carboxymethyl cellulose hydrogel composite synthesized using K. Hansenii NQ4 with the addition of 4% medium viscosity carboxymethyl cellulose (scale bar 100 μm).

FIG. 5 is a first order red polarized light image of a bacterial cellulose/carboxymethyl cellulose hydrogel composite synthesized using K. Hansenii NQ4 with the addition of 4% medium viscosity carboxymethyl cellulose (scale bar 100 μm).

FIG. 6 is a polar extinction image of a bacterial cellulose/carboxymethyl cellulose hydrogel composite synthesized using K. Hansenii NQ4 with the addition of 4% medium viscosity carboxymethyl cellulose (scale bar 100 μm).

FIG. 7 is a bright field image of a bacterial cellulose/carboxymethyl cellulose hydrogel composite synthesized using K. Hansenii NQ5 with the addition of 1% medium viscosity carboxymethyl cellulose (scale bar 100 μm).

FIG. 8 is a first order red polarized light image of a bacterial cellulose/carboxymethyl cellulose hydrogel composite synthesized using K. Hansenii NQ5 with the addition of 1% medium viscosity carboxymethyl cellulose (scale bar 100 μm).

FIG. 9 is a polar extinction image of a bacterial cellulose/carboxymethyl cellulose hydrogel composite synthesized using K. Hansenii NQ5 with the addition of 1% medium viscosity carboxymethyl cellulose (scale bar 100 μm).

FIG. 10 is a bright field image of a bacterial cellulose/carboxymethyl cellulose hydrogel composite synthesized using K. Hansenii NQ5 with the addition of 4% medium viscosity carboxymethyl cellulose (scale bar 100 μm).

FIG. 11 is a first order red polarized light image of a bacterial cellulose/carboxymethyl cellulose hydrogel composite synthesized using K. Hansenii NQ5 with the addition of 4% medium viscosity carboxymethyl cellulose (scale bar 100 μm).

FIG. 12 is a polar extinction image of a bacterial cellulose/carboxymethyl cellulose hydrogel composite synthesized using K. Hansenii NQ5 with the addition of 4% medium viscosity carboxymethyl cellulose (scale bar 100 μm).

FIG. 13 is an image of a bacterial cellulose/carboxymethyl cellulose hydrogel composite synthesized using K. Hansenii NQ4 with the addition of 1% medium viscosity carboxymethyl cellulose taken using phase contrast microscopy setting on the microscope condenser and an objective that does not have the phase plate where the condenser annulus (ring) projects a cone of light around the specimen creating the shadowing effect on the surface (scale bar 100 μm).

FIG. 14 is an image of a bacterial cellulose/carboxymethyl cellulose hydrogel composite synthesized using K. Hansenii NQ4 with the addition of 4% medium viscosity carboxymethyl cellulose taken using phase contrast microscopy setting on the microscope condenser and an objective that does not have the phase plate where the condenser annulus (ring) projects a cone of light around the specimen creating the shadowing effect on the surface (scale bar 100 μm).

FIG. 15 is an image of a bacterial cellulose/carboxymethyl cellulose hydrogel composite synthesized using K. Hansenii NQ5 with the addition of 1% medium viscosity carboxymethyl cellulose taken using phase contrast microscopy setting on the microscope condenser and an objective that does not have the phase plate where the condenser annulus (ring) projects a cone of light around the specimen creating the shadowing effect on the surface (scale bar 100 μm).

FIG. 16 is an image of a bacterial cellulose/carboxymethyl cellulose hydrogel composite synthesized using K. Hansenii NQ5 with the addition of 4% medium viscosity carboxymethyl cellulose taken using phase contrast microscopy setting on the microscope condenser and an objective that does not have the phase plate where the condenser annulus (ring) projects a cone of light around the specimen creating the shadowing effect on the surface (scale bar 100 μm).

FIG. 17 is a 0.8×BF image of the right hand before bacterial cellulose gel application.

FIG. 18 is a 0.8×BF dissection scope image of the right hand 30 minutes after bacterial cellulose gel application.

FIG. 19 is a 0.8×BF dissection scope image of the right hand one hour after bacterial cellulose gel application.

FIG. 20 is a 1.0× dissection scope image of the left hand control before lotion application.

FIG. 21 is a 1.0× dissection scope image of the left hand 30 minutes after lotion application.

FIG. 22 is a 1.0× dissection scope image of the left hand pre-lotion treatment (trial 2).

FIG. 23 is a 1.0× dissection scope image of the left hand 30 minutes after lotion application (trial 2).

FIG. 24 is a 1.0× dissection scope image of the right hand before carboxymethyl cellulose bacterial cellulose gel application (trial 2).

FIG. 25 is a 1.0× dissection scope image of the right hand 24 hours after carboxymethyl cellulose bacterial cellulose gel application (trial 2).

FIG. 26 is a 1.0× dissection scope image of the right hand before carboxymethyl cellulose bacterial cellulose gel treatment (trial 3).

FIG. 27 is a 1.0× dissection scope image of the right hand 30 minutes after carboxymethyl cellulose bacterial cellulose gel application.

FIG. 28 is a 1.0× dissection scope image of the right hand 24 hours after carboxymethyl cellulose bacterial cellulose gel application.

### DETAILED DESCRIPTION

The compositions and methods described herein may be understood more readily by reference to the following detailed description of specific aspects of the disclosed subject matter and the Examples included therein.

Before the present compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid the reader in distinguishing the various components, features, or steps of the disclosed subject matter. The identifiers “first” and “second” are not intended to imply any particular order, amount, preference, or importance to the components or steps modified by these terms.

Disclosed herein are methods of making composite cellulose hydrogels, the methods comprising providing a cellulose synthesizing microbe; and culturing the cellulose synthesizing microbe in a composition comprising greater than 1% of a cellulose derivative thereby forming the composite cellulose hydrogel.

As used herein, a “hydrogel” indicates a three-dimensional polymeric network that is highly hydrophilic (e.g., they can contain over 99.9% water) and capable of maintaining its structural integrity.

As used herein, a “cellulose synthesizing microbe” is any microbe capable of synthesizing cellulose. The cellulose synthesizing microbe can be one or more prokaryotic organisms capable of generating cellulose, for example, Salmonella, Agrobacterium, Rhizobium, Nostoc, Scytonema, Anabaena, Acetobacter, Gluconacetobacter, or Komagataeibacter. In some examples the cellulose synthesizing microbe comprises a species of Komagataeibacter, such as Komagataeibacter hansenii. In some examples, the cellulose synthesizing microbe can comprises the NQ5 strain of Komagataeibacter hansenii (ATCC 53582) and/or the NQ4 strain of Komagataeibacter hansenii.

The gram negative bacterium, Komagataeibacter hansenii (formerly Gluconacetobacter xylinus; Acetobacter xylinum), is a particularly efficient producer of pure, highly crystalline cellulose, bacterial cellulose (BC) (Nishi Y et al. J Mater Sci. 1990, 25, 2997-3001; Cousins S K and Brown R M Jr. Polymer. 1997, 38, 903-913; Nobles D and Brown R M Jr. Cellulose. 2008, 15, 691-701). Bacterial cellulose has an ultra-fine reticulated structure, high crystallinity, great mechanical strength, high water holding capacity, moldability during formation, and biocompatibility (Yamanaka S et al. J Mater Sci. 1989, 24, 3141-3145; Ross P et al. Microbiol. Rev. 1991, 55, 35-58; Yoshinaga F et al. Biosci. Biotechnol. Biochem. 1997, 61, 219-224; Czaja W et al. Cellulose. 2004, 11, 403-411).

The cellulose synthesizing microbe can be cultured according to known methods using standard culture conditions. The culture conditions can be varied, for example, to affect the dimensions and/or properties of the composite cellulose hydrogel. In some examples, the cellulose synthesizing microbe can be cultured under agitated culture conditions. The cellulose synthesizing microbe can be cultured for an amount of time of 2 days or more (e.g., 3 days or more, 4 days or more, 5 days or more, 6 days or more, 7 days or more, 8 days or more, 9 days or more, 10 days or more, 11 days or more, 12 days or more, or 13 days or more). In some examples, the cellulose synthesizing microbe can be cultured for an amount of time of 14 days or less (e.g., 13 days or less, 12 days or less, 11 days or less, 10 days or less, 9 days or less, 8 days or less, 7 days or less, 6 days or less, 5 days or less, 4 days or less, or 3 days or less). The amount of time that the cellulose synthesizing microbe is cultured can range from any of the minimum values described to any of the maximum values described above. For example, the cellulose synthesizing microbe can be cultured for an amount of time from 2 days to 14 days (e.g., from 2 days to 8 days, from 8 days to 14 days, from 2 days to 5 days, from 5 days to 8 days, from 8 days to 11 days, from 11 days to 14 days, or from 4 days to 12 days).

The cellulose synthesizing microbe can be cultured in a composition comprising any appropriate nutrient media. Examples of appropriate nutrient media include standard nutrient media such as GYC which contains (g/liter of distilled water): yeast extract, 10.0; D-glucose, 50.0; CaCO₃, 30.0 and agar, 25.0. Various alternatives such as replacements for glucose or yeast extract, and omissions of agar or CaCO₃ are usable and well-known to those skilled in the art (Bergey's Manual of Systematic Biology, Vol. 1 pp 268-276, Krieg, ed. Williams and Wilkins, Baltimore/London (1984)). One useful nutrient medium used directly or with modifications described herein was that first described by Schramm and Hestrin (Hestrin, et al., Biochem. J. Vol. 58 pp 345-352 (1954). Standard Schramm Hestrin (SH) medium contains (g/L): D-glucose, 20; peptone, 5; yeast extract, 5; dibasic sodium phosphate, 2.7, and citric acid monohydrate, 1.15 (pH adjusted to between about 3.5 and 5.5 with HCl). When SH is used without glucose (SH-gluc), this indicates the above SH composition, but without the 10 g glucose/liter addition.

The cellulose synthesizing microbe can be cultured in a composition comprising greater than 1% of the cellulose derivative (e.g., 1.5% or more, 2% or more, 2.5% or more, 3% or more, 3.25% or more, 3.5% or more, 3.75% or more, 4% or more, 4.25% or more, 4.5% or more, 4.75% or more, 5% or more, or 5.5% or more). In some examples, the cellulose synthesizing microbe can be cultured in a composition comprising 6% or less of the cellulose derivative (e.g., 5.5% or less, 5% or less, 4.75% or less, 4.5% or less, 4.25% or less, 4% or less, 3.75% or less, 3.5% or less, 3.25% or less, 3% or less, 2.5% or less, 2% or less, or 1.5% or less). The amount of the cellulose derivative in the composition the cellulose synthesizing microbe is cultured in can range from any of the minimum values described above to any of the maximum values described above. For example, the cellulose synthesizing microbe can be cultured in a composition comprising from greater than 1% to 6% of the cellulose derivative (e.g., from 2% to 6%, from 2.5% to 5.5%, from 3% to 5%, from 3.25% to 2.75%, from 3.5% to 4.5%, or from 3.75% to 4.25%). In some examples, the cellulose synthesizing microbe can be cultured in a composition comprising 4% of the cellulose derivative.

The cellulose derivative can, for example, comprise any cellulosic material that can increase the water holding capacity of the microbial cellulose, alter the moldability of the microbial cellulose, or otherwise alter the mechanical properties of the microbial cellulose. For example, the cellulose derivative can be selected from the group consisting of carboxymethyl cellulose, methylcellulose, hydroxyethyl cellulose, hydroxypropyl cellulose, hydroxypropylmethyl cellulose, and combinations thereof. In some examples, the cellulose derivative is carboxymethyl cellulose.

In some examples, the methods can further comprise isolating the composite cellulose hydrogel. Isolating the composite cellulose hydrogel can comprise, for example, centrifugation, filtration, or a combination thereof.

In some examples, the methods can further comprise rinsing the composite cellulose hydrogel. For example, the composite cellulose hydrogel can be rinsed with water. In some examples, the methods can further comprise sterilizing the composite cellulose hydrogel. The composite cellulose hydrogel can, for example, be sterilized by autoclaving.

Also disclosed herein are the composite cellulose hydrogels made by the methods described herein. For example, the composite cellulose hydrogels can comprise a gel. The composite cellulose hydrogels described herein can, for example, be biocompatible. As used herein, the term “biocompatible” means that there is minimal (i.e., no significant difference is seen compared to a control), if any, effect on the surroundings of the location in a body where the composite cellulose hydrogel is placed.

Also disclosed herein are articles of manufacture comprising the composite cellulose hydrogels described herein. Examples of articles of manufacture include, but are not limited to, wound dressings, subdermal fillers, tissue scaffolds, drug delivery agents, topical dermal repair agents, and combinations thereof.

The wound dressings can, for example, be placed on the surface of the wound or into the wound bed. This wound healing system can augment the effective regeneration of new tissues in situ in the body. The wound dressings can be used for a wide variety of wound types, locations, shapes, depth and stage(s) of healing.

As used herein, the term “wound” is used to refer broadly to injuries to the skin and subcutaneous tissue initiated in different ways (e.g., pressure sores from extended bed rest and wounds induced by trauma) and with varying characteristics. Wounds are generally classified into one of four grades depending on the depth of the wound: Grade I: wounds limited to the epithelium; Grade II: wounds extending into the dermis; Grade III: wounds extending into the subcutaneous tissue; and Grade IV (or full-thickness wounds), which are wounds in which bones are exposed (e.g., a bony pressure point such as the greater trochanter or the sacrum). As used herein, the term “partial thickness wound” refers to wounds that encompass Grades I-III; e.g., burn wounds, pressure sores, venous stasis ulcers, and diabetic ulcers. As used herein, the term “deep wound” is used to describe to both Grade III and Grade IV wounds. As used herein, the term “chronic wound” refers to a wound that has not healed within 30 days.

As used herein, the term “dressing” refers broadly to the composite cellulose hydrogels when prepared for, and applied to, a wound for protection, absorbance, drainage, etc. The wound dressings described herein can further include any one of the numerous types of backings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (non-woven composites of fibers from calcium alginate), and cellophane (cellulose with a plasticizer).

In most applications, the wound dressing comprising the composite cellulose hydrogels will be sterilized and can also be formed into a suture, a sheet, a compress, a bandage, a band, a prosthesis, a fiber, a woven fiber, a bead, a strip, a gauze or combinations thereof. The wound dressing comprising the composite cellulose hydrogels can also include a portion that is self-adhesive and/or an adhesive backing. The wound dressing comprising the composite cellulose hydrogels can, in some examples, be formed into a dressing that is molded to fit a specific wound site.

In some examples, the articles of manufacture include implantable articles of manufacture, e.g., articles of manufacture that can be implanted. For example, the article of manufacture can comprise a biocompatible implant that comprises the composite cellulose hydrogels.

As used herein, the term “implanted” is used to describe the positioning of the composite cellulose hydrogel in the wound,” e.g., by contacting some part of the wound with the composite cellulose hydrogel. As used herein, the term “integrated” is used to describe the temporary, semi-temporary, semi-permanent or permanent integration of the composite cellulose hydrogel as part of the healed portion of a wound. The composite cellulose hydrogel can become semi- or permanently integrated as part of the final healed site because it is non-immunogenic. In some forms, the composite cellulose hydrogel serves as a scaffold for the migration and growth of new cells at the wound site during and even after the entire healing process if the composite cellulose hydrogel is allowed to remain. Generally, at least part of the composite cellulose hydrogel will remain in the wound site as it becomes an integral part of the scar tissue.

Also disclosed herein are methods of use of the composite cellulose hydrogels described herein. For example, the methods of use of the composite cellulose hydrogels can comprise methods of treating a wound. As used herein, the phrases “promote wound healing,” “enhance wound healing,” and the like refer to either the induction of the formation of granulation tissue of wound contraction and/or the induction of epithelialization (i.e., the generation of new cells in the epithelium) by the composite cellulose hydrogels described herein.

The cellulose composite hydrogels and/or the wound dressings comprising the composite cellulose hydrogels can, for example, be used in the treatment of chronic wounds, ulcers, facial masks, and other wound sites. Furthermore, the wound dressings comprising the composite cellulose hydrogels can be used for the treatment of all types of wounds, e.g., those caused by laser surgery, chemical burns, cancer treatments, biopsy excision sites, scars from pathogens, entry wounds, cosmetic surgery, reconstructive surgery and the like.

In some examples, the composite cellulose hydrogels can be used to treat a wound wherein the wound comprises a cutaneous wound. Examples of cutaneous wounds include, but are not limited to, burn wounds, neuropathic ulcers, pressure sores, venous stasis ulcers, and diabetic ulcers.

The most traumatic and complex of all skin injuries are caused by burns, and this results in an extensive damage to the various skin layers. Burns are generally defined according to depth and range from 1st degree (superficial) to 3rd degree (entire destruction of epidermis and dermis). The standard protocol of burn management highlights several factors which accelerate the process of optimal healing: (a) control of fluid loss; (b) barrier to wound infection; (c) fast and effective wound closure, optimally with skin grafts or skin-substitutes; and, (d) significant pain relief.

In some examples, the composite cellulose hydrogels can be used to treat a wound wherein the wound comprises a chronic wound. Chronic wounds such as venous leg ulcers, bedsores, and diabetic ulcers are difficult to heal, and they represent a significant clinical challenge both to the patients and to the health care professionals. Wounds that do not heal readily can cause the subject considerable physical, emotional, and social distress as well as great financial expense. Wounds that fail to heal properly and become infected often require excision of the affected tissue.

The method of treating the wound can comprise applying the composite cellulose hydrogel to the wound. The composite cellulose hydrogel can, for example, be applied to the wound for an amount of time of 1 hour or more (e.g., 2 hours or more, 3 hours or more, 6 hours or more, 12 hours or more, 18 hours or more, 24 hours or more, 36 hours or more, 2 days or more, or 1 week or more).

In some examples, the composite cellulose hydrogel can be used for tissue regeneration by injecting the composite cellulose hydrogel into the tissue in need to regeneration. The injected composite cellulose hydrogel can, for example, provide a scaffold for the integration of cells necessary for regeneration within the tissue.

In some examples, the composite cellulose hydrogels described herein can be used in environmental applications, such as for moisture retention, soil erosion prevention, and the like.

The composite cellulose hydrogels described herein can also be used for drug delivery applications. For example, the composite cellulose hydrogels can be used to control drug delivery to a site through controlling diffusion at the site.

Another method of use of the composite cellulose hydrogels described herein are as food additives. For example, the composite cellulose hydrogels can be used in a food of dietary item.

Another method of use of the composite cellulose hydrogels described herein are as cosmetic dermal filler.

The examples below are intended to further illustrate certain aspects of the methods and compounds described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

Cellulose is a crystalline biopolymer comprised of extended chains of β-1,4-linked glucose residues; it is an abundant bio-macromolecule that is produced by plants, algae, tunicates, colorless protists, as well as photosynthetic and heterotrophic bacteria. The gram negative bacterium, Komagataeibacter hansenii (formerly Gluconacetobacter hansenii, Gluconacetobacter xylinus, Acetobacter xylinum), is a particularly efficient producer of a pure, highly crystalline cellulose called bacterial cellulose (BC). Bacterial cellulose has distinctive properties that differentiate it from cellulose found in other organisms and is particularly well suited for medical, industrial, and commercial applications because of its ultra-fine reticulated structure, high crystallinity, mechanical strength, high water holding capacity, moldability during formation, and biocompatibility.

Traditionally, the main focus of study has been on the utilization of bacterial cellulose membranes for various applications; however, further study into manipulations during synthesis whereby the addition of certain reagents results in the alteration of the final cellulose product may broaden the array of possible applications for bacterial cellulose. Previous studies have shown that the structure and hierarchical cell-directed self-assembly process of cellulose found in K. hansenii make it more amenable to such manipulations during synthesis. The biosynthesis of cellulose in K. hansenii occurs as a consecutive, linked two-step process. The first step involves the polymerization of glucose residues within the catalytic sites of the cellulose synthesizing protein complex to form polymer chains. The second step occurs when van der Waals forces facilitate the crystallization of the polymer chains into glucan mini-sheets. The mini-sheets undergo hydrogen bonding to form cellulose mini-crystals that exit the pore complex. The crystallization step continues external to the cell whereby the nascent cellulose mini-crystals associate into microfibrils, the microfibrils associate into bundles, and the bundles aggregate into the final ribbon.

The external crystallization step is where the addition of certain outside reagents has the most influence. The fluorescent brightener Tinopal LPW (4,4′-bis[2-hydroxyethylamino-1,3,5-triazin-2-yliamino]-2,2′-stilbenedisulfonic acid, previously referred to as Calcofluor White™) was demonstrated to interrupt the in vivo assembly of crystalline cellulose I microfibrils in Acetobacter xylinum by competing for the hydrogen bonding sites within the glucose residues of the nascent glucan mini-crystals. This interruption produced cellulose in the form of broad bands of bent fibrils that were non-crystalline and half the size (15 Å) of wild type microfibrils (30 Å).

Sodium alginate (NaAgl) addition to the culture medium of Acetobacter xylinum NUST4.1 was determined to increase cellulose production, accelerate growth during early phase cell division, and alter bacterial cellulose morphology through hydrogen bonding during the cellulose biosynthesis process. The resulting cellulose had a net-like cellulose mesh appearance that was covered with particles of sodium alginate.

Carboxymethyl cellulose (CMC) was used as a chemical probe to interrupt the last step of cellulose assembly in Acetobacter xylinum ATCC 23769 by inhibiting the integration of bundles of cellulose I microfibrils into ribbons. The resulting cellulose pellicles were thinner and more fragile than control membranes. Additionally, it was determined that under agitated conditions, when G. xylinus was incubated with carboxymethyl cellulose, a disorganized “slime” or hydrogel composite consisting of fine filaments of bacterial cellulose intertwined with carboxymethyl cellulose was produced instead of a durable aggregate of cellulose.

Carboxymethyl cellulose is a form of cellulose that is generated by the insertion of carboxymethyl groups along the polymer backbone allowing it to be soluble in water. An important factor when considering the production of a bacterial cellulose/carboxymethyl cellulose hydrogel composite is the bonding scheme created by the degree of substitution (DS). The degree of substitution refers to the number of carboxymethyl groups attached to the free hydroxyls found on the carboxymethyl cellulose glucose unit. Each carboxymethyl cellulose glucose unit has three free hydroxyl groups that have the capacity to bond to another cellulose backbone. If the degree of substitution is 3, then all three hydroxyls would be shielded by the carboxymethyl groups from bonding. A degree of substitution of 0.4, 0.7, or 1.2 would allow for more of the free carboxymethyl cellulose hydroxyl groups to associate with the cellulose backbone from another source such as native cellulose produced by G. xylinus. Haigler et al. determined that a degree of substitution of 0.7 was the most effective at disrupting the final step in the hierarchical cell-directed self-assembly process in G. xylinus whereby the bundles of microfibrils were not allowed to associate to form the final ribbon assembly (Haigler C H et al. J. Cell Biol. 1982, 94, 64-69). Furthermore, once the carboxymethyl cellulose coats the bundles of microfibrils, subsequent hydrogen bonding between the native cellulose is prevented through steric hindrance or electrostatic repulsion as the coated cellulose is now neutral or charged thereby assuring the production of a hydrogel composite.

On the basis of this analysis, the effects of the addition of carboxymethyl cellulose in different concentrations and viscosities to agitated cultures of K. hansenii for the purposes of producing a bacterial cellulose/carboxymethyl cellulose hydrogel composite were studied. A tunable cellulose bio-nanocomposite hydrogel with unique structural and mechanical properties was created for possible use in a wide range of biomedical, industrial, or commercial applications.

### Preparation of the Bacterial Cellulose/Carboxymethyl Cellulose Hydrogel Composite Cell Inoculum

To obtain a high concentration cellulose solution for inoculation, K. hansenii ATCC 53582 strain NQ5 and K. hansenii strain NQ4 (Laboratory Stock) were grown for 4 days in test tubes containing 10 mL Schramm and Hestrin (SH) medium (Schramm M and Hestrin S. J Gen Microbial, 1954, 11, 123-9) consisting of (per liter): 20.0 g of glucose (Fisher D16-10), 5.0 g of bacto peptone (BD 211820), 5.0 g bacto yeast extract (BD 212720), 2.7 g of sodium phosphate dibasic heptahydrate (Fisher 7782-85-6), and 1.5 g of citric acid (Mallinckrodt 0627-12) at 28° C. under static conditions. Pellicles from each strain were harvested and placed in two 500 ml flasks containing 100 ml SH supplemented with 0.8% Celluclast (cellulase). The flasks were placed on a rotary shaker set at 140 rpms and cultured for 5 days or until the cellulose was completely broken down. The resulting cell solution was harvested by using a centrifugation washing process whereby the cells were spun at 3300 rpm for 10 minutes, supernatant discarded, resuspended in 50 mL of Acetobacter buffer (5.1 g/L Sodium Phosphate and 1.15 g/L Citric Acid), spun for another 10 minutes, washed again, and finally resuspended in 20 mL of the Acetobacter buffer. Cell inoculum concentration of OD₆₀₀ of 2 was determined by spectrophotometry.

### Preparation of the Microbial Cellulose/Low, Medium, and High Viscosity Carboxymethyl Cellulose Hydrogel Composites

The bacterial cellulose/carboxymethyl cellulose hydrogel composites were produced by inoculating 2 L Erlenmeyer flasks containing 500 mL of SH medium and supplemented with 0%, 1%, 2%, 3%, and 4% low, medium, or high viscosity carboxymethyl cellulose (Sigma Aldrich C-5678) with 1.5 mL of the inoculum. The flasks were placed on a rotary shaker set at 140 rpm and allowed to culture for 7 days. The resulting cellulose was harvested and cleaned by rinsing with deionized H₂O (dH₂O), suspended in a washing solution of 2% Contrex AP (Decon Labs), autoclave sterilized, shaken overnight, rinsed again, and sterilized a final time by autoclaving.

The results of the addition of carboxymethyl cellulose (degree of substitution 0.7; low viscosity) to agitated cultures of K. hansenii NQ4 and NQ5 (agitated at 140 rpm; flask size 500 mL; media volume 100 mL; cultured for 7 days) are shown in Table 1.

TABLE 1
K. hansenii NQ4 and NQ5 Bacterial cellulose/Carboxymethyl
cellulose hydrogel properties with agitation at 140 rpm.

| | Wet | Dry | | |
|---|---|---|---|---|
| | weight | weight | Swelling | |
| Sample | (g) | (g) | ratio | Notes: morphology |
| NQ4 | 143.4 | 1.46 | 98.2 | aggregate of strong cellulose |
| control | | | | |
| NQ4 1% | 151.6 | 1.55 | 97.8 | almost cellulose pellets |
| NQ4 2% | 149.5 | 1.51 | 99.0 | globular gel |
| NQ4 3% | 125.8 | 1.29 | 97.5 | a more uniform thick gel but |
| | | | | still with globular texture |
| NQ4 4% | 98.2 | 0.99 | 99.2 | uniform gel |
| NQ5 | 158.6 | 1.6 | 99.1 | aggregate of strong cellulose |
| control | | | | |
| NQ5 1% | 174.8 | 1.78 | 98.2 | almost cellulose pellets |
| NQ5 2% | 159.7 | 1.61 | 99.2 | globular gel |
| NQ5 3% | 143.7 | 1.45 | 99.1 | a more uniform thick gel but |
| | | | | still with globular texture |
| NQ5 4% | 99.4 | 1 | 99.4 | uniform gel |

The results of the addition of carboxymethyl cellulose (degree of substitution 0.7; low viscosity) to agitated cultures of K. hansenii NQ4 and NQ5 (agitated at 80 rpm; flask size 500 mL; media volume 100 mL; cultured for 7 days) are shown in Table 2.

TABLE 2
K. hansenii NQ4 and NQ5 Bacterial cellulose/Carboxymethyl
cellulose hydrogel properties with agitation at 80 rpm.

| | Wet | Dry | | |
|---|---|---|---|---|
| | weight | weight | Swelling | |
| Sample | (g) | (g) | ratio | Notes: morphology |
| NQ4 control | 139.6 | 1.45 | 96.3 | aggregate of strong |
| | | | | cellulose/almost a pellicle |
| NQ4 1% | 156.3 | 1.59 | 98.3 | aggregate of weak |
| | | | | cellulose that |
| | | | | almost formed a pellicle |
| NQ4 2% | 139.5 | 1.43 | 97.6 | in between an aggregate |
| | | | | pellicle and a gel |
| NQ4 3% | 122.6 | 1.25 | 98.1 | thick hydrogel |
| NQ4 4% | 101.2 | 1.04 | 97.3 | hydrogel |
| NQ5 control | 160.7 | 1.67 | 96.2 | aggregate of strong |
| | | | | cellulose/almost a pellicle |
| NQ5 1% | 180.9 | 1.84 | 98.3 | aggregate of weak |
| | | | | cellulose that |
| | | | | almost formed a pellicle |
| NQ5 2% | 162.4 | 1.66 | 97.8 | in between an aggregate |
| | | | | pellicle and a gel |
| NQ5 3% | 145.8 | 1.49 | 97.9 | thick hydrogel |
| NQ5 4% | 104.6 | 1.06 | 98.7 | hydrogel |

The results of the addition of carboxymethyl cellulose (degree of substitution 0.7) of low, medium, and high viscosity to agitated cultures of K. hansenii NQ4 (agitated at 140 rpm; flask size 2000 mL; media volume 500 mL; cultured for 7 days) are shown in Table 3.

TABLE 3
K. hansenii NQ4 Bacterial cellulose/Carboxymethyl cellulose hydrogel properties
with the addition of low, medium and high viscosity carboxymethyl cellulose.

| | | Dry | | Volume of | |
|---|---|---|---|---|---|
| CMC | Wet | weight | Swelling | hydrogel | |
| viscosity | weight (g) | (g) | ratio | (mL) | Notes: morphology |
| low | 1337.5 | 17.5 | — | 250 | Fibrous gel with larger lumps |
| medium | 473 | 4.83 | 97.9 | 100 | Smooth gel but volume |
| | | | | | greatly reduced |
| high | 908 | 9.15 | 99.2 | 200 | Fibrous gel/small clumps of |
| | | | | | BC-CMC?/membrane on top |
| | | | | | after 7 days |

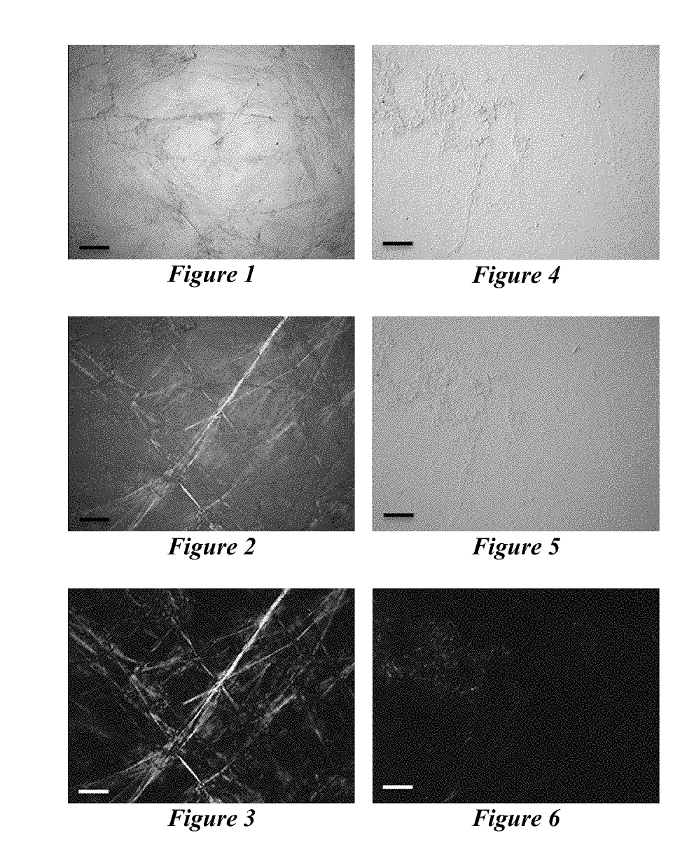

A bright field image, a first order red polarized light image, and a polar extinction image of a bacterial cellulose/carboxymethyl cellulose hydrogel composite synthesized using K. Hansenii NQ4 with the addition of 1% medium viscosity carboxymethyl cellulose are shown in FIG. 1, FIG. 2, and FIG. 3, respectively. A bright field image, a first order red polarized light image, and a polar extinction image of a bacterial cellulose/carboxymethyl cellulose hydrogel composite synthesized using K. Hansenii NQ4 with the addition of 4% medium viscosity carboxymethyl cellulose are shown in FIG. 4, FIG. 5, and FIG. 6, respectively. The birefringence in the first order red polarized light image and polar extinction images for the bacterial cellulose/carboxymethyl cellulose hydrogel composite synthesized using the lower concentration of carboxymethyl cellulose (FIG. 2 and FIG. 3) indicates a more crystalline structure than for the bacterial cellulose/carboxymethyl cellulose hydrogel composite synthesized using the higher concentration of carboxymethyl cellulose.

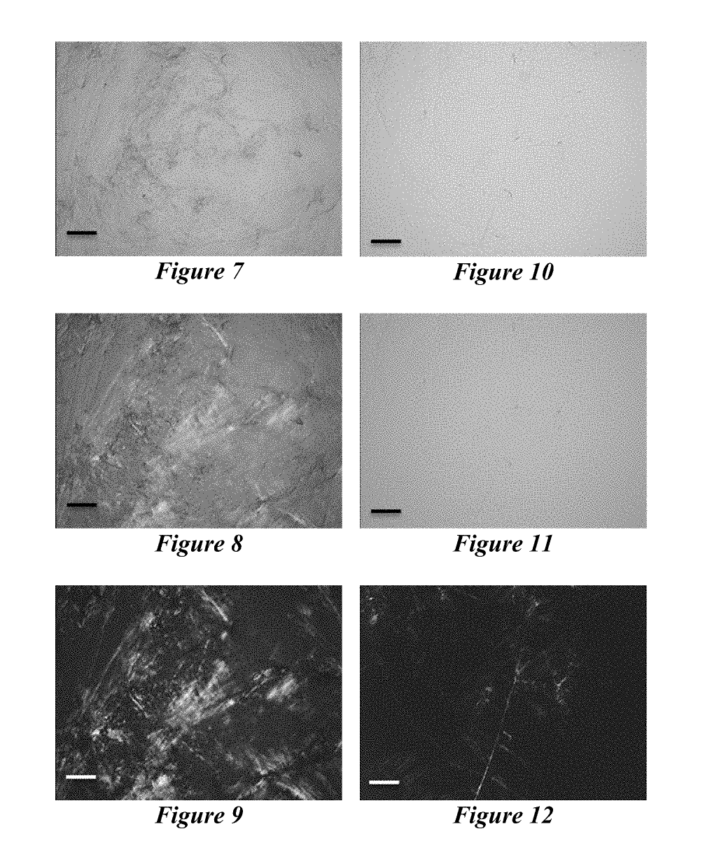

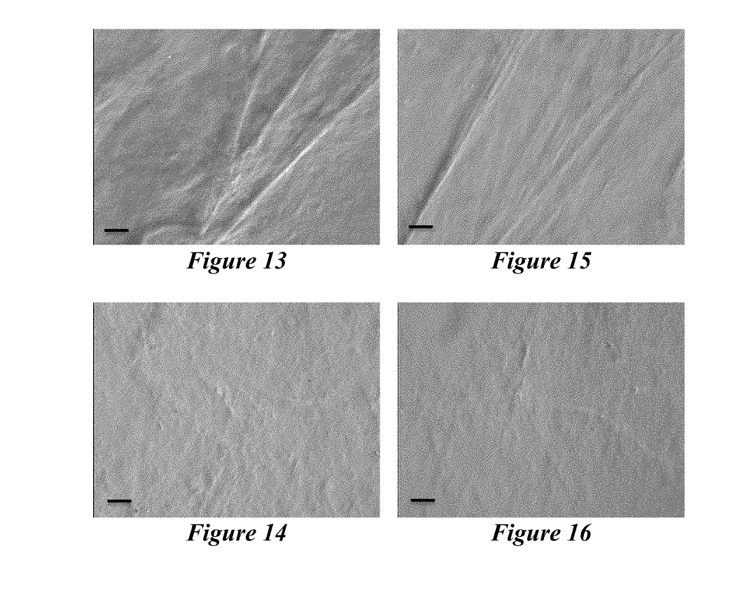

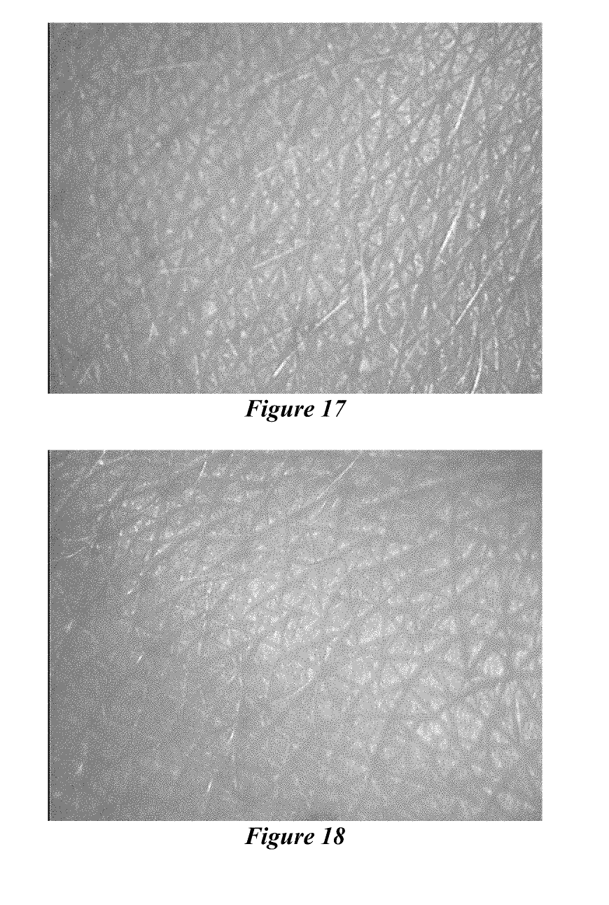

A bright field image, a first order red polarized light image, and a polar extinction image of a bacterial cellulose/carboxymethyl cellulose hydrogel composite synthesized using K. Hansenii NQ5 with the addition of 1% medium viscosity carboxymethyl cellulose are shown in FIG. 7, FIG. 8, and FIG. 9, respectively. A bright field image, a first order red polarized light image, and a polar extinction image of a bacterial cellulose/carboxymethyl cellulose hydrogel composite synthesized using K. Hansenii NQ5 with the addition of 4% medium viscosity carboxymethyl cellulose are shown in FIG. 10, FIG. 11, and FIG. 12, respectively. The birefringence in the first order red polarized light image and polar extinction images for the bacterial cellulose/carboxymethyl cellulose hydrogel composite synthesized using the lower concentration of carboxymethyl cellulose (FIG. 8 and FIG. 9) indicates a more crystalline structure than for the bacterial cellulose/carboxymethyl cellulose hydrogel composite synthesized using the higher concentration of carboxymethyl cellulose.

The surface of various gels were imaged using phase contrast microscopy setting on the microscope condenser and an objective that does not have the phase plate. The condenser annulus (ring) projects a cone of light around the specimen creating the shadowing effect on the surface. Images of samples of a bacterial cellulose/carboxymethyl cellulose hydrogel composite synthesized using K. Hansenii NQ4 with the addition of 1% medium viscosity carboxymethyl cellulose, a bacterial cellulose/carboxymethyl cellulose hydrogel composite synthesized using K. Hansenii NQ4 with the addition of 4% medium viscosity carboxymethyl cellulose, a bacterial cellulose/carboxymethyl cellulose hydrogel composite synthesized using K. Hansenii NQ5 with the addition of 1% medium viscosity carboxymethyl cellulose, and a bacterial cellulose/carboxymethyl cellulose hydrogel composite synthesized using K. Hansenii NQ5 with the addition of 4% medium viscosity carboxymethyl cellulose are shown in FIGS. 13-16, respectively.

### Example 2

Microbial cellulose, also known and bacterial cellulose (BC) can be manipulated to obtain different characteristics depending on the desired uses. Bacterial cellulose (BC), a cellulose membrane produced by a bacterial organism during a fermentation process, is currently used in the food, beauty, and engineering industries. The bacteria culture is added to SH nutrient media and grown in specific time periods to produce a membrane or, as discussed herein, a gel. The bacteria form spinnerets and produce bundles of fibers known as fibrils, which together form a bacterial cellulose membrane. Bacterial cellulose can absorb up to 100% its own weight of water and has great tensile strength.

Herein, the ability of cellulose to be used in cosmetics to increase product effectiveness and alleviate the cosmetics industry of environmentally harmful ingredients is explored. A bacterial cellulose gel of set consistency and yield was synthesized using lab techniques for growing cellulose to increase viscosity for cosmetic uses.

The carboxymethyl cellulose bacterial cellulose composite gels were made using the procedures described above in Example 1, except different concentrations of carboxymethyl cellulose were used. To add the carboxymethyl cellulose into solution, 500 ml of deionized water was added to six 1 liter beakers, followed by varying concentrations of carboxymethyl cellulose ranging from 0% (control) to 6%. For the 1% carboxymethyl cellulose sample, 5 grams of carboxymethyl cellulose was added; for the 2% carboxymethyl cellulose sample, 10 grams of carboxymethyl cellulose was added; for the 3% carboxymethyl cellulose sample, 15 grams of carboxymethyl cellulose was added; for the 4% carboxymethyl cellulose sample, 20 grams of carboxymethyl cellulose was added; for the 5% carboxymethyl cellulose sample, 25 grams of carboxymethyl cellulose was added; and for the 6% carboxymethyl cellulose sample, 30 grams of carboxymethyl cellulose was added.

The 4% carboxymethyl cellulose sample had the smoothest consistency and highest yield in both trials. Based on this result, additional experiments were performed using 3.75%, 4% and 4.25% carboxymethyl cellulose using the same techniques as described above.

It was observed that increasing the carboxymethyl cellulose concentration past 4% resulted in a decrease in viscosity. Samples with lower than 4% carboxymethyl cellulose produced a cellulose membrane in addition to the hydrogel. The 4% carboxymethyl cellulose concentration samples gave the smoothest consistency and highest yield of any of the samples tested. The yield was nearly double the 3.75% and 4.25% carboxymethyl cellulose concentration samples. 4% carboxymethyl cellulose samples yielded 50-75 ml in all 3 trials, while the other samples yielded less than 50 ml. The carboxymethyl cellulose bacterial cellulose gel's viscosity was increased by adding 2.5 milliliters of carbomer, a thickening agent used in foods, cosmetics, and miscellaneous fluids.

### Example 3

The bacterial cellulose gel described above can also be used for cosmetic purposes. Several thickeners are used in makeup today; the ability to replace those thickeners with the bacterial cellulose gel discussed herein was examined. For example, the use of bacterial cellulose gel can be as a cosmetic facial mask alternative to conventional bacterial cellulose sheet masks was studied. Bacterial cellulose can interact with the extracellular matrix of human skin, for example, increasing the moisture barrier, healing the extracellular matrix of human skin, and acting as a nutrient serum vector. One benefit, for example, of using a gel over a membrane is the amount of time the material can be worn. A membrane dries out over a 30-minute time span whereas the gel can be worn overnight, thereby works with the skin for a greater duration of time, which can increase the results and nutrient absorption. As discussed above, the viscosity of the bacterial cellulose gel for a specific cosmetic purpose can be enhanced with a small amount of carbomer.

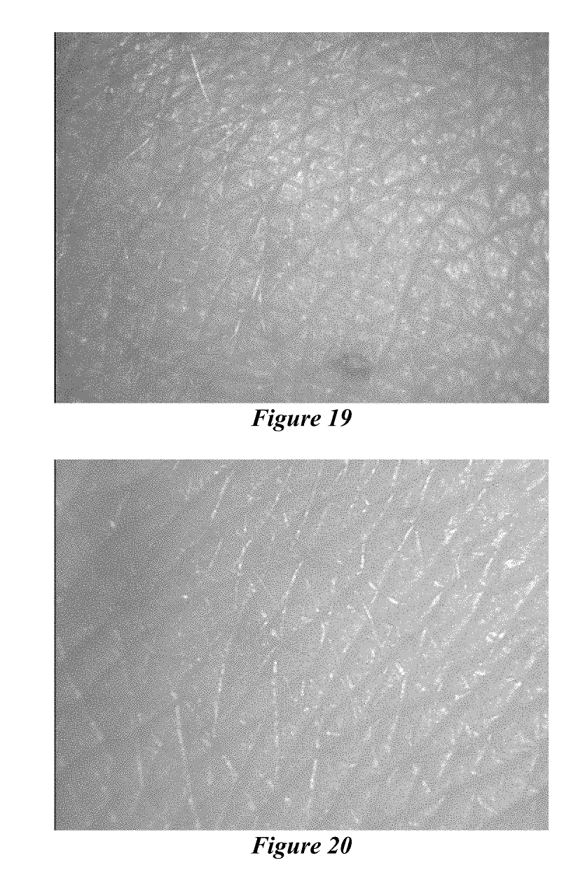

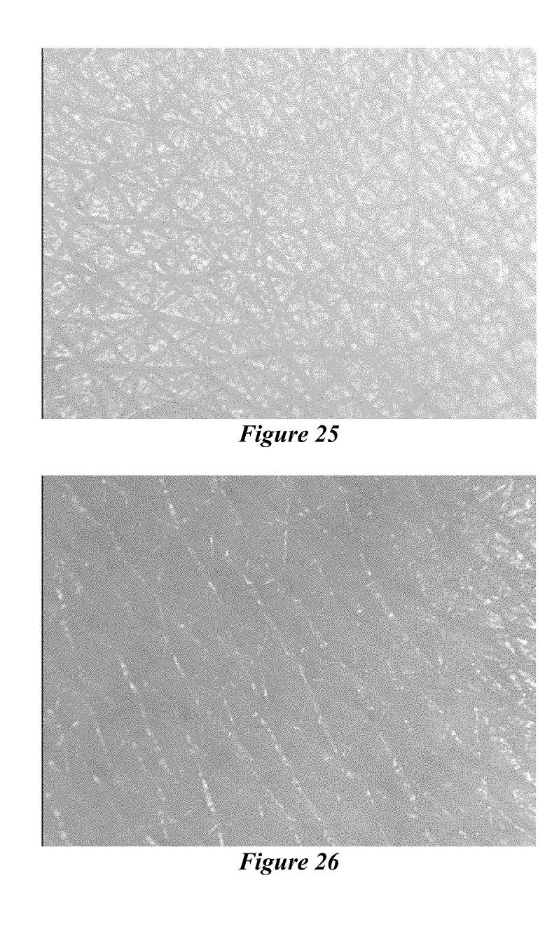

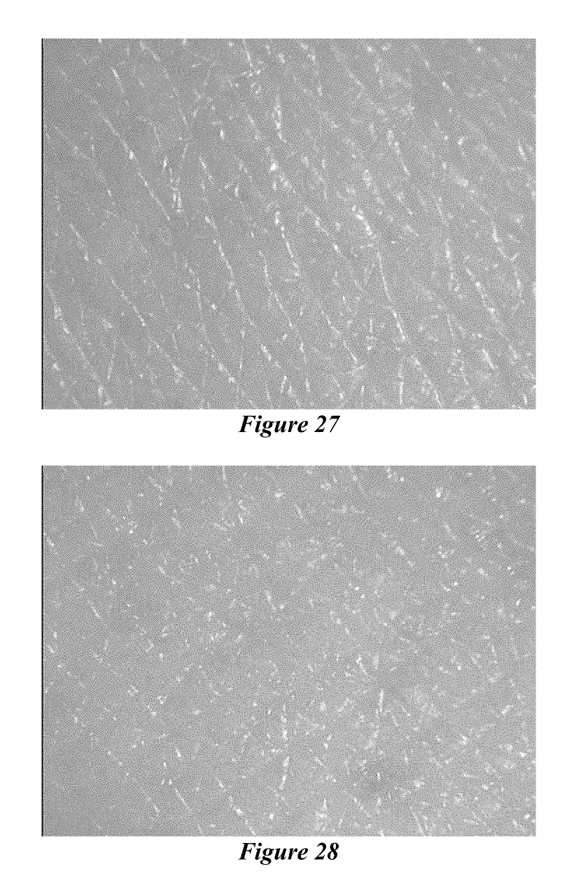

The interaction of the bacterial cellulose gel with the extracellular matrix of human skin was investigated. To test the effects of bacterial cellulose gel on the skin, the bacterial cellulose gel was applied to the back of a human subject's hand and the surface of the treated skin was then compared to the surface of the skin on the opposite untreated hand. The effects of the carboxymethyl cellulose bacterial cellulose gel on the skin were examined using light microscopy to analyze any skin changes 30 minutes and 24 hours after application. The microscope gel hand trials showed that the skin became smoother and more relaxed (moisturized) after application of the carboxymethyl cellulose bacterial cellulose gel, compared to the control hand (FIG. 17-FIG. 19; FIG. 24-FIG. 28).

Since lotion is commonly used for increasing moisture of skin, experiments were also performed to compare the effect of commercially available lotions with the bacterial cellulose gel treatment. The lotion used in these experiments was Loccitane Rose Hand Cream, considered to be a high quality and effective skin moisturizer. For these comparative lotion experiments, lotion and bacterial cellulose gel treatments were applied to the back of a human subject's hand and the differences in the appearance of the skin with a lotion treatment was compared to a bacterial cellulose gel treatment in 30 minute and 24 hour intervals. More specifically, the left hand was used for the control (lotion) sample and the right hand was used for the experimental bacterial cellulose gel sample. Camera images were obtained and analyzed under the dissection microscope before and after each product dried (˜30 minutes each). The carboxymethyl cellulose bacterial cellulose gel was viewed using bright field microscopy to analyzed the gel structure and compared it to other cellulose forms previously studied.

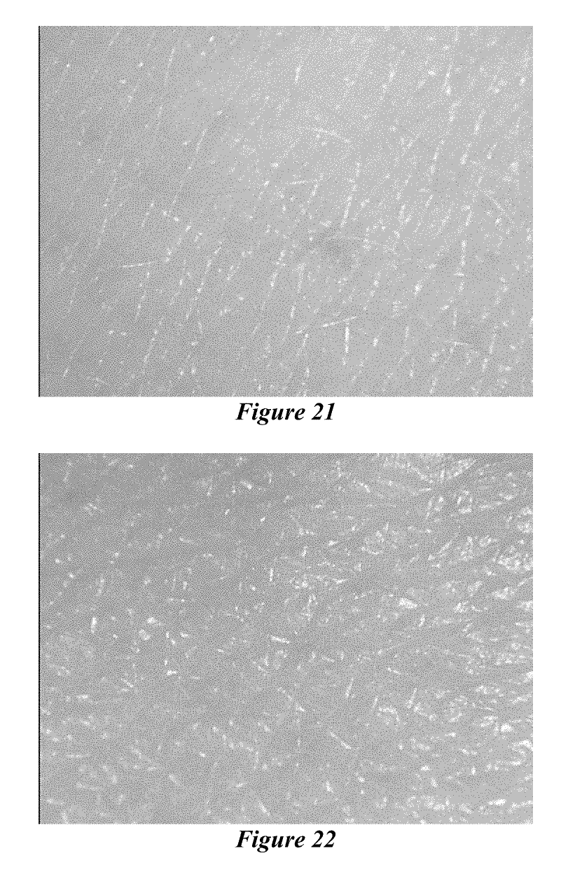

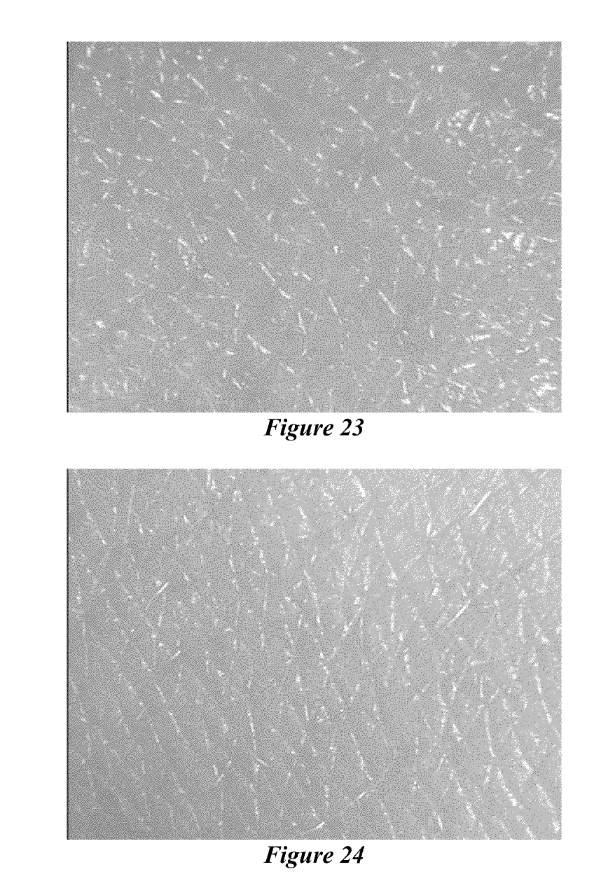

The results from the 30 minute and 24 hour hand trials show a positive interaction between the cellulose and the skin (FIG. 17-FIG. 19; FIG. 24-FIG. 28). The skin appears to have greater smoothness, suppleness and tone evenness. The lotion trials also improved the appearance of the skin (FIG. 20-FIG. 23); however the results appear to not be as intense or long lasting in comparison with the carboxymethyl cellulose bacterial cellulose gel trials.

The 4% carboxymethyl cellulose bacterial cellulose composite gel has great promise for cosmetic uses. The gel can be used to enhance cosmetic product's effectiveness and skin smoothness. A variety of cosmetics including primers, creams, serums and liquid/cream foundations can be created using the bacterial cellulose gel in appropriate concentrations. Bacterial cellulose sheet masks are growing in popularity in the US. Bacterial cellulose sheet masks originated and are commonly used in South Korea for their positive effects on the skin. Bacterial cellulose sheet masks are reported to increase the moisture barrier of the skin, heal, decrease irritation, temporarily reduce the appearance of fine lines, and even the skin tone. A few US dermatologists are using bacterial cellulose sheet masks to heal and reduce irritation in post procedure skin (chemical peel, dermabrasion, etc.). As discussed earlier, a benefit of a bacterial cellulose gel mask is the increased duration of time the mask can be worn for; the gel mask could be worn overnight to decrease irritation and healing time post-procedure. The overnight bacterial cellulose gel mask could work to increase moisture and allow nutrients to enter the skin in a time-released manner. Another application of the carboxymethyl cellulose bacterial cellulose gel would be to relieve the pain and damage associated with sunburns.

The difference in structure and effectiveness of the bacterial cellulose gel compared to NQ5 cellulose membranes and carboxymethyl cellulose NQ5 cellulose membrane hybrids was also investigated. The methods involved drying NQ5 cellulose membranes and NQ5 carboxymethyl cellulose membrane hybrids and testing their characteristics for packaging and sterilization for biomedical applications, such as wound dressing. The sheets dried in 3 days on a glass surface and fit in a standard autoclave pouch. The samples did not break down from the heat or pressure of the autoclave, allowing them to become sterilized without losing material.

Cellulose can act as a tissue scaffold, making cellulose of interest for use in biomedical applications such as in wound dressings (Svensson et al. Biomaterials, 2005, 24(4), 419-431). Bacterial cellulose can be used as a bandage and burn treatment, in some examples, resulting in rapid healing and minimal scaring. The bacterial cellulose gel can be used for deep wound healing that a stand-alone cellulose bandage would not work for.

### Example 4

An example of a cosmetic use for bacterial cellulose gel is in sub-dermal fillers such as those seen in cosmetic dermatology. There are currently no bacterial cellulose based sub-dermal fillers on the market. The industry standards for sub-dermal fillers are hyaluronic acid (HA) and collagen injections from bovine sources. Occasionally, the body rejects these fluids causing an adverse reaction that requires invasive procedures to negate the effects or causes the patient to wait until their body metabolizes the fluid. The human body does not contain antibodies for cellulose, so there would be no potential adverse reaction upon injection of a bacterial cellulose based sub-dermal filler. Furthermore, the bacterial cellulose gel could last longer than current injectable fillers due to the human body's lack of cellulases (enzymes that break down cellulose). Additionally, the bacterial cellulose gel filler could be removed, if desired, by a non-invasive method such as a cellulase injection. Cellulase cannot break down human tissue and would only break down the filler, leaving the area in a pre-injection state.

Cellulose has previously been studied for use as a tissue scaffold in mice for tissue regeneration of all types. Carboxymethyl cellulose bacterial cellulose is a possible solution for deep wound healing, as it could act as a filler and scaffold at the same time. Filling the wound with an oxygen permeable gel would help seal the wound while promoting healing, for example by recruiting healing factors necessary for proper healing.

The 4% carboxymethyl cellulose bacterial cellulose gel was also analyzed for possible cosmetic filler properties. For the bacterial cellulose gel to be an appropriate alternative cosmetic filler, it needs to fit through a small needle. The needle size commonly used in for standard cosmetic fillers is 29½. Accordingly, the carboxymethyl cellulose bacterial cellulose gel was tested with a 22 and 29½ gauge needle. As it would be aesthetically inefficient for a cosmetic filler to move easily, the carboxymethyl cellulose bacterial cellulose gel was also tested to see how well the carboxymethyl cellulose bacterial cellulose gel adhered to surfaces. To test the adhesion, a syringe was used to drop small droplets of the carboxymethyl cellulose bacterial cellulose gel onto the surface of a stretched latex glove. Then, the glove was moved in different orientations and directions. Bacterial cellulose gels with other carboxymethyl cellulose concentrations were not tested for possible cosmetic filler properties due to their thinner consistency and low level of surface adherence.

The carboxymethyl cellulose bacterial cellulose composite gel fit easily through the 29½ gauge needle. The testing on the adhesion level of the 4% carboxymethyl cellulose sample found that the carboxymethyl cellulose bacterial cellulose gel droplets on the latex surface did not move when manually disturbed, in all directions and orientations tested. The carboxymethyl cellulose bacterial cellulose gel droplets did not move until they were manually wiped off of the glove surface.

Non-invasive facial rejuvenation options are available in injectable filler form through most cosmetic dermatologists. Research on sub-dermal cosmetic fillers has been done on cross-linked carboxymethyl cellulose hydrogels. The study reported positive results with the filler trials. However, their process involves cross-linking the cellulose to obtain a gel (Leonardis et al. 2015). The bacterial cellulose gels discussed herein do not require cross-linking due to the addition of carboxymethyl cellulose and growth under agitated conditions. The processes discussed herein are more time and cost effective while producing a similar gel to the cross-linked cellulose hydrogel. The SH media used in each 4% carboxymethyl cellulose bacterial cellulose gel sample flask costs $3.45 to produce, making it a more affordable option than hyaluronic acid (HA)/collagen fillers to produce.

The carboxymethyl cellulose bacterial cellulose gel fit through an appropriate size needle for cosmetic injectable use (e.g., 29½ gauge needle). The carboxymethyl cellulose bacterial cellulose gel can be used in the field of cosmetics at the skin's surface and sub-dermal layers.

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions and methods, and aspects of these compositions and methods are specifically described, other compositions and methods and combinations of various features of the compositions and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

What is claimed is:

1. A method of making a composite cellulose hydrogel, comprising:
providing a cellulose synthesizing microbe, wherein the cellulose synthesizing microbe comprises Komagataeibacter hansenii; and
culturing the cellulose synthesizing microbe in a composition comprising greater than 1% of a cellulose derivative, thereby forming the composite cellulose hydrogel.

2. The method of claim 1, wherein the cellulose synthesizing microbe comprises the ATCC 53582 NQ5 strain of Komagataeibacter hansenii.

3. The method of claim 1, wherein the cellulose synthesizing microbe comprises the NQ4 strain of Komagataeibacter hansenii.

4. The method of claim 1, wherein the cellulose synthesizing microbe is cultured under agitated culture conditions.

5. The method of claim 1, wherein the cellulose synthesizing microbe is cultured for an amount of time from 2 to 14 days.

6. The method of claim 1, wherein the concentration of the cellulose derivative in the composition is from 2% to 6%.

7. The method of claim 1, wherein the concentration of the cellulose derivative in the composition is from 3% to 5%.

8. The method of claim 1, wherein the concentration of the cellulose derivative in the composition is from 3.75% to 4.25%.

9. The method of claim 1, wherein the cellulose derivative is selected from the group consisting of carboxymethyl cellulose, methylcellulose, hydroxyethyl cellulose, hydroxypropyl cellulose, hydroxypropylmethyl cellulose, and combinations thereof.

10. The method of claim 1, wherein the cellulose derivative is carboxymethyl cellulose.

11. The method of claim 1, wherein the method further comprises isolating the composite cellulose hydrogel.

12. The method of claim 11, wherein isolating the composite cellulose hydrogel comprises filtration, centrifugation, or a combination thereof.

13. A composite cellulose hydrogel made by the method of claim 1.

14. An article of manufacture comprising the composite cellulose hydrogel made by the method of claim 1.

15. The article of manufacture of claim 14, wherein the article of manufacture comprises a wound dressing, a subdermal filler, a tissue scaffold, a drug delivery agent, a topical dermal repair agent, or combinations thereof.

16. A method of use of the composite cellulose hydrogel made by the method of claim 1, the method comprising treating a wound.

17. The method of claim 16, wherein the wound comprises a cutaneous wound.

18. The method of claim 16, wherein the wound comprises a chronic wound.

19. The method of claim 16, wherein the method of treating the wound comprises applying the composite cellulose hydrogel to the wound.

20. The method of claim 19, wherein the composite cellulose hydrogel is applied to the wound for an amount of time of 1 hour or more.
